(19) United States
(12) Patent Application Publication
(10) Pub. No.: US 2014/0128912 A1
(43) Pub. Date: May 8, 2014

(54) Tungsten-Rhenium Alloys for Curved Surgical Needle Applications

(71) Applicant: Ethicon, Inc., Somerville, NJ (US)

(72) Inventors: Thomas Wilkes, New Brunswick, NJ (US); Frank Cichocki, Easton, PA (US)

(73) Assignee: Ethicon, Inc., Somerville, NJ (US)

(21) Appl. No.: 14/055,079

(22) Filed: Oct. 16, 2013

Related U.S. Application Data
(60) Provisional application No. 61/721,591, filed on Nov. 2, 2012.

(51) Int. Cl. A61B 17/06

(52) U.S. Cl. CPC A61B 17/06066
USPC 606/222

(57) ABSTRACT

Curved surgical needles made from tungsten rhenium alloys are disclosed. The curved surgical needles have body flats. The needles have improved resistance to cracking.

### FIELD OF THE INVENTION

The present invention relates to refractory metal alloys, more specifically, tungsten-rhenium alloys for use in surgical needles.

### BACKGROUND OF THE INVENTION

Surgical needles are well known in the art. Surgical needles in combination with attached surgical sutures are used in conventional surgical procedures for a variety of applications, including approximating tissue, affixing implants to tissue, connecting blood vessels, lifting or displacing tissue, etc. Surgical needles are typically elongated members having distal pointed tissue piercing ends and proximal suture mounting ends. The distal ends may also have cutting edges formed into the needle body to assist the passage of the needle through tissue. The proximal suture mounting ends may have various suture mounting structures including drilled bore holes and formed channels. The distal ends of surgical sutures are mounted in the bore holes or channels, and attached by various conventional methods including mechanical swaging, gluing, etc.

It is well known that surgery places stringent requirements on suture needles. Both the needle strength and stiffness must be great enough to withstand the forces necessary to penetrate tissue. At the same time, the needle must be ductile enough to prevent brittle failure and allow the surgeon to reshape the device should it flex from the original shape during use.

Surgical needles are conventionally made from high-strength martensitic stainless steel alloys such as AISI type 420, ASTM 45500 and ETHALLOY®. ETHALLOY® is a proprietary alloy of Ethicon, Inc. used to make surgical needles. The beneficial combination of strength, stiffness and ductility of these alloys is attributed to their alloying additions and processing parameters. More recently, refractory metals and their alloys have gained attention due to their superior mechanical properties. Such alloys, including tungsten-rhenium alloys, are disclosed in U.S. Pat. No. 5,415,707 to Bendel et al., which is incorporated by reference.

Fine diameter needles made from fine diameter wires are often used in demanding, critical surgical procedures such as coronary artery by-pass graft (CABG) procedures associated with open heart surgery. In such procedures it is critical that the needle perform while maintaining its structural integrity. It is known that in a CABG procedure, the surgeon may encounter situations where the patient has calcified plaque in the coronary arteries subject to graft by-pass. Conventional stainless steel needles may bend when the surgeon attempts to push the needle through the calcified plaque mass. This may present problems in that the needle may deform or bend in a non-elastic manner. Such bending typically renders the needle unusable, requiring the surgeon to discard the bent needle and halt the procedure in order to acquire a new surgical needle and suture to complete the procedure. Excessive bending of the needle may also alter the pathway of the needle through the tissue, possibly complicating the surgical procedure.

The use of refractory metal alloys to manufacture surgical needles provides a fine diameter surgical needle that has improved stiffness and is less susceptible to bending in a surgical procedure. However, it is known that there may be challenges in manufacturing surgical needles from refractory metal alloys such as tungsten rhenium. Surgical needles in order to be useful in surgery are typically curved. The curvature may vary, for example, from ⅛ of a circle to ⅝ of a circle. In addition, surgical needles typically have flattened side sections, or “flats”. The flats are useful for grasping of the needle by the surgeon using conventional needle grasper instruments in order to prevent the needle body from rotating in the grasper. Without flats, curved suture needles can easy rotate inadvertently during use resulting in possible tissue damage or at the least a high level of frustration for the surgeon. This is a problem that is unique to curved suture needles as forces that may be generated at the needle point easily generate a twisting moment that can cause curved suture needles lacking adequate body flats to suddenly flip or rotate in the needle grasper. These same forces that may be generated at the point of a straight suture needle do not easily cause the needle to suddenly flip or re-orient in the needle grasper in a manner resulting in an untoward translation motion of the needle point that can cause damage to tissue or frustration to the surgeon. The flats and other structures formed into the needle body may also provide structural advantages with respect to resisting bending, column buckling, etc.

There is a continuing need in this art for novel surgical needles having improved properties while also having conventional features such as curved configurations and flats.

### SUMMARY OF THE INVENTION

A curved surgical needle is disclosed. The needle has a curved elongated body having a distal end with a piercing point and a proximal surgical suture mounting end, and has at least one lateral flat along at least part of the body. The flat has a surface. The needle is made from a refractory metal alloy containing about 70 wt. % to less than about 95 wt. % of tungsten and greater than about 5 wt. % to about 30 wt. % rhenium, and having a breaking strength of about 450 ksi to about 590 ksi. The surgical needle is resistant to cracking.

Another aspect of the present invention is a curved surgical needle. The needle has a curved elongated body having a distal end with a piercing point and a proximal surgical suture mounting end, and has at least one lateral flat along at least part of the body. The flat has a surface. The needle is made from a refractory metal alloy containing about 74 wt. % to about 85 wt. % of tungsten and from about 15 wt. % to about 26 wt. % rhenium, and having a breaking strength of about 450 ksi to about 590 ksi. The surgical needle is resistant to cracking.

These and other aspects and advantages of the present invention will become more apparent from the following description and accompanying drawings.

### BRIEF DESCRIPTION OF THE DRAWINGS

FIG. 1 is a flow diagram illustrating the general processes used to make curved surgical needles of Example 1.

FIG. 2 is an optical micrograph of surgical needles with body flats made from tungsten-rhenium alloys as described in Example 1

FIG. 3 is a graph illustrating the bending performance (ASTM1874) of surgical needles without body flats made from tungsten-rhenium alloys containing either 5, 15 or 26 wt. % rhenium.

FIG. 4 is a graph illustrating the bending performance (ASTM1874) of surgical needles with body flats made from tungsten-rhenium alloys containing either 15 or 26 wt. % rhenium and a conventional high strength stainless steel alloy.

FIG. 5 is a drawing depicting a surgical needle and body flat geometry with X and T dimensions as described in Example 1.

### DETAILED DESCRIPTION OF THE INVENTION

The refractory metal alloys of the present invention useful for making curved surgical needles that are resistant to cracking will be an alloy of tungsten and rhenium. The alloys will typically contain about 70 wt. % to about less than 95 wt. % of tungsten, more typically about 74 wt. % to about less than 95 wt. %, and preferably 74 wt. % to about 85 wt. %. The alloys will additionally contain rhenium, typically in an amount of about greater than 5 wt. % to about 30 wt. %, more typically greater than 5 wt. % to about 26 wt. %, and preferably about 15 wt. % to about 26 wt. %. The alloys may also contain trace amounts of other elements including iron (Fe) and molybdenum (Mo). The trace amounts will be present in an amount of less than about 0.05 wt. %, and preferably less than about 0.03 wt. %.

The curved surgical needles of the present invention made from the alloys of the present invention will be made utilizing conventional needles manufacturing techniques that may be modified when using refractory metal alloys. Once the wire is drawn to the desired final diameter using conventional wire drawing and hot-working techniques, conventional techniques are used to apply a desired point shape to one end of the needle. Flats may be applied to the body of the needle by conventional pressing applications that form the body into a variety of shapes. Although the needles may have one flat applied, it is generally preferred to have two or more body flats. The needle may then be curved to the desired radius of curvature. Methods of curving surgical needles made from refractory metal alloys are disclosed in U.S. Pat. No. 7,937,981 which is incorporated by reference. In the proximal end of the needle, a structure is formed to facilitate the mounting of a suture. This may be done using conventional techniques including swaging or adhesive attachment. Needles having flats and methods of incorporating flats into surgical needles are described in U.S. Pat. Nos. 3,160,157 and 4,799,484, both of which are incorporated by reference in their entirety. The flats may optionally contain ribs extending up from the surface of the flats.

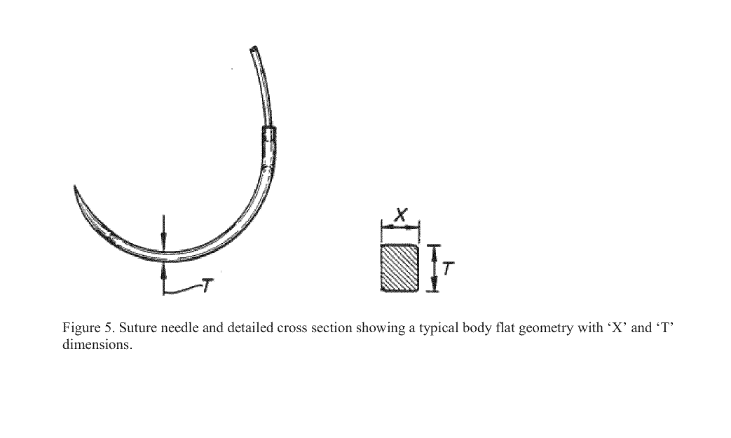

It is particularly preferred that the flats in the surgical needles of the present invention have a T to X ratio of typically about 1.0 to about 1.2, preferably about 1.0 to 1.1. FIG. 5 illustrates a needle having T and X dimensions. The T dimension is seen to be the height of the needle body cross-section while the X dimension is the width. The dimensions T and X scale with the diameter of the wire from which the needle is made. Typically, the dimensions are taken at the mid-point of the needle, although depending on the overall configuration of the needle, the T and X ratios may be taken at other points along the needle body. It should be noted that although the section in FIG. 5 is shown to have body flats on four sides, the needles may also have opposed body flats on two sides with the remaining surfaces retaining the curved surfaces of the needle wire. It is also possible to have a body flat on just one side. In this ratio, the absolute value of the T and X dimensions will depend on the diameter of the wire from which the needle is made and then to a lesser extent on the degree of electropolishing that is conducted after machine forming of the suture needle blank has been made. By way of example, a suture needle produced from wire with an incoming diameter of 0.0105 inches may nominally exhibit a T=0.0094 inches and X=0.0090 inches resulting in a T to X ratio of approximately 1.04. A ratio of greater than 1.0 is desired in order to gain a bending moment boost. However, a ratio of less than 1.0 is undesirable because the bending strength of the needle will be reduced. A ratio of greater than about 1.2 is undesirable because the needle will be less stable when armed across the top and bottom flats due to the reduction in flat surface area that occurs at high T to X ratios.

Curved surgical needles are required to have a number of properties in order to provide a desirable range of function. The stiffness of the needle should be such that elastic deflection is resisted allowing for a high level of control and placement of the needle and suture. A relatively high bending stiffness results in a relatively low tendency for flexure and deformation during use. ASTM standard F1874-98 (2011) details the Standard Method for Bend Testing of Needles Used in Surgical Sutures. The ASTM outlines measurement of the yield moment and maximum (ultimate) moment in bending. These two properties are measures of needle strength, with greater values being preferred.

In addition to the stiffness and strength of a needle, ductility is of the utmost importance. If any part of the needle is too brittle, it may break during use without bending. A common measure of needle ductility is bending the needle through an angle of 90 degrees and then returning the needle to the original curvature. This reshaping process simulates deformation during use and the ability to reshape and continue using the needle as opposed to halting a surgical procedure to acquire a new needle. The more reshapes a needle can withstand without breaking, the more ductile it is. A numerical reshape value of 1.0 is defined as the needles being able to withstand both the initial 90 degree deformation and the reshaping of the needle to the original curvature. Should the needle withstand another 90 degree deformation without breaking, the needle is said to have a reshape ductility of 1.5. Subsequently, should the needle again withstand reshaping to the original curvature without breaking, the needle has a reshape ductility of 2.0. This process is repeated, and 0.5 increments are added to the reshape value, until the needle breaks.

The following example is illustrative of the principles and practice of the present invention although not limited thereto.

### EXAMPLE 1

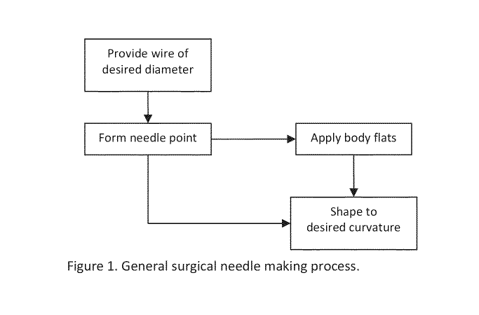

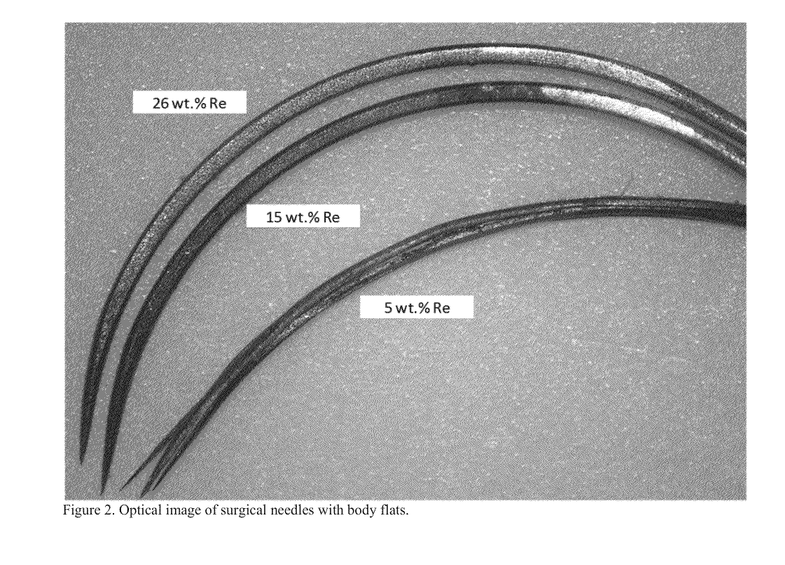

Surgical needles were produced using conventional methods described earlier in this disclosure and in the above-cited United States patents. FIG. 1 illustrates the general process flow resulting in surgical needles with or without body flats. FIG. 2 depicts 0.008 inch diameter surgical needles with body flats made from tungsten-rhenium alloys containing rhenium in the amount of either 5 wt. %, 15 wt. % or 26 wt. %. Marked fracturing occurred (as seen in FIG. 2) when using the tungsten-5 wt. % rhenium alloy.

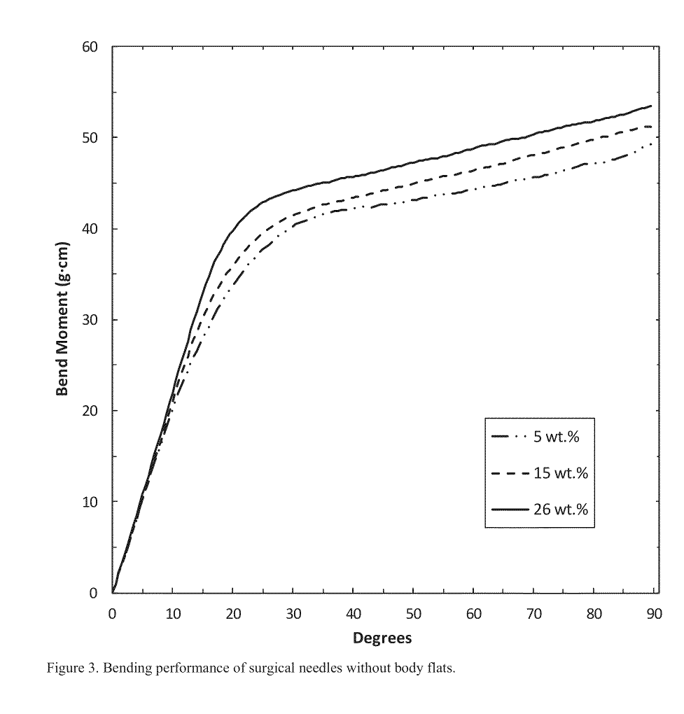

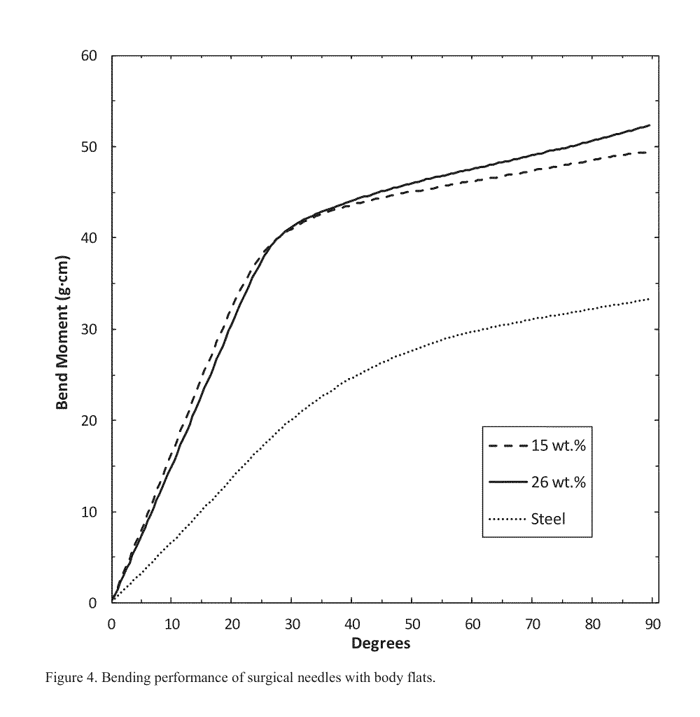

The curves in FIG. 3 illustrate the bending performance of needles without flats made from tungsten-rhenium alloys containing about 5 wt. %, 15 wt. %, and 26 wt. % rhenium. The curves in FIG. 4 illustrate the bending performance of needles with flats made from tungsten-rhenium alloys containing about 15 wt. % and 26 wt. % rhenium. No significant compromise in bending performance was made by decreasing the rhenium content of the alloy used in making round-bodied surgical needles; however, a rhenium content of only about 5 wt. % precluded the processing of a surgical needle with body flats. Each of the curves in FIGS. 3 and 4 was an average of the data from at least 5 individual samples.

The novel alloys and curved surgical needles of the present invention made from these alloys have many advantages. The use of a refractory tungsten rhenium alloy in the ranges described results in a superior surgical needle compared to conventional stainless steel needles. The combination of high strength and high stiffness is complemented by high ductility within the disclosed alloy ranges. This combination of mechanical properties results in curved surgical needles having at least one body flat that are resistant to cracking.

Although this invention has been shown and described with respect to detailed embodiments thereof, it will be understood by those skilled in the art that various changes in form and detail thereof may be made without departing from the spirit and scope of the claimed invention.

We claim:

1. A curved surgical needle, comprising:
a curved elongated body having a distal end with a piercing point and a proximal surgical suture mounting end, and having at least one lateral flat along at least part of the body, the flat having a surface, wherein the needle comprises:
a refractory metal alloy, said alloy comprising,
about 70 wt. % to less than about 95 wt. % of tungsten; and,
greater than about 5 wt. % to about 30 wt. % of rhenium, wherein the alloy has a strength of about 450 ksi to about 590 ksi,
wherein the curved surgical needle is resistant to cracking.

2. The surgical needle of claim 1 wherein the body flat has a T to X ratio of the lateral flat is about 1.0 to about 1.2.

3. The surgical needle of claim 2, wherein the T to X ratio is about 1.0 to about 1.1.

4. The surgical needle of claim 1, wherein the alloy comprises greater than about 5 wt. % to about 26 wt. % or rhenium.

5. The surgical needle of claim 1, additionally comprising at least one rib member extending from the surface of the flat.

6. The surgical needle of claim 1 having a reshape value of at least 1.0.

7. A curved surgical needle, comprising:
a curved elongated body having a distal end with a piercing point and a proximal surgical suture mounting end, and having at least one lateral flat along at least part of the body, the flat having a surface wherein the needle comprises:
a refractory metal alloy, said alloy comprising,
about 74 wt. % to about 85 wt. % of tungsten; and,
about 15 wt. % to about 26 wt. % of rhenium, wherein the alloy has a breaking strength of about 450 ksi to about 590 ksi,
wherein the curved surgical needle is resistant to cracking.

8. The surgical needle of claim 7 wherein the body flat has a T to X ratio of about 1.0 to about 1.2.

9. The surgical needle of claim 8, wherein the T to X ratio is about 1.0 to about 1.1.

10. The surgical needle of claim 7, additionally comprising at least one rib member extending from the surface of the flat.

11. The surgical needle of claim 7 having a reshape value of at least 1.0.
